(19) United States
(12) Patent Application Publication
(10) Pub. No.: US 2026/0224685 A1
(43) Pub. Date: Aug. 6, 2026

(54) A NOVEL mRNA VACCINE FOR THE TREATMENT AND PREVENTION OF HPV-ASSOCIATED LESIONS AND TUMORS

(71) Applicant: VIROGIN BIOTECH (SHANGHAI) LTD., Shanghai (CN)

(72) Inventors: Kuan Zhang, Shanghai (CN); Zhenglong Wang, Shanghai (CN); Yin Liu, Shanghai (CN); Dong Zuo, Shanghai (CN); Jun Ding, Vancouver (CA); William Wei-Guo Jia, Vancouver (CA); Zhibin Yu, Shanghai (CN)

(21) Appl. No.: 19/146,196

(22) PCT Filed: Jul. 13, 2023
(86) PCT No.: PCT/CN2023/107199

§ 371 Date: Jul. 7, 2025

(30) Foreign Application Priority Data
Jan. 5, 2023 (WO) PCT/CN2023/070634

(51) Int. Cl. A61K 39/12; A61K 39/00; A61P 37/00; C07K 14/005

(52) U.S. Cl. CPC A61K 39/12; A61P 37/00; C07K 14/005; A61K 2039/54; C07K 2319/00

(57) ABSTRACT

Immunogenic proteins are provided which have been derived from a strain of HPV which causes or is associated with causing cancer, which are mutated so that they do not cause increased cell proliferation. In various embodiments, the immunogenic protein is derived (e.g., mutated) from an HPV strain such as HPV 16, 18, 31, 33,35, 39, 45, 51, 52, 56, 58, 59, 66 or 68. Also provided are compositions (e.g., mRNA compositions) encoding such immunogenic proteins, as well as method for treating and preventing HPV infections.

### FIELD OF THE INVENTION

The present invention relates generally to compositions and methods for treating and/or preventing human papillomavirus (HPV)-infections and associated lesions or tumors. REFERENCE TO SEQUENCE LISTING, TABLE OR COMPUTER PROGRAM

The contents of the electronic sequence listing (VIRO422PC_SEQLISTING.xml; Size: 19,952 bytes; and Date of Creation: Jul. 13, 2023) is herein incorporated by reference in its entirety.

### BACKGROUND

Human papillomavirus or “HPV” is a DNA virus from the Papillomaviridae family which is the most common sexually transmitted virus in the world. Over ninety percent of infections resolve spontaneously, but in some cases the infections can persist for years as warts or precancerous lesions. In some instances, particularly for infections of the cervix, vulva, vagina, penis, anus, mouth, tonsils and throat, lesions can develop into cancer. Although there are over 200 strains of HPV, 70% of cancers are most frequently caused by HPV strains type 16 and 18.

A number of vaccines are currently available to prevent infection by some strains of HPV, including GARDASIL, GARDASIL 9, and CERVARIX. GARDASIL is a quadrivalent vaccine against HPV types 6, 11, 16 and 18. GARDASIL 9 protects against HPV types 6, 11, 16, 18, 31, 33, 45, 52, and 58 (the latter covering an additional 20% of cervical cancers not covered by the quadrivalent vaccine).

Although the current available HPV vaccines generate protective antibodies that effectively prevent HPV infection, they don't produce effective T cell responses to clear HPV infected cells or HPV-related cancer cells. The HPV infected population is not protected by those vaccines and urgently needs an effective treatment.

### SUMMARY

The present invention provides immunogenic proteins or nucleotide sequences encoding such proteins that can be used to treat and/or prevent HPV-related diseases, including HPV-associated skin disease, HPV-related tumor development and to cause HPV-related tumor regression via strong T cell responses.

Within certain embodiments of the invention, compositions and methods are provided comprising one or more immunogenic proteins which have been derived from a strain of HPV which causes or is associated with causing cancer, which are mutated so that they do not cause increased cell proliferation. Within various embodiments of the invention the immunogenic protein is derived from an HPV strain which is associated with causing cancer or the proliferation of cells in subjects may be similarly mutated. Representative examples include, but are not limited to HPV strains 16, 18, 31, 33, 35, 39, 45, 51, 52, 56, 58, 59, 66 and 68.

Within further embodiments the immunogenic protein may be fused to an immune cell targeting moiety such as HA protein in order to increase its utility as a vaccine.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Features of the present disclosure, its nature and various advantages will be apparent from the accompanying drawings and the following detailed description of various embodiments.

FIGS. 1A, 1B, and 1C present the consensus amino acid sequences of E6(16) (SEQ ID NO: 1) and E7(16) (SEQ ID NO:2), the amino acids mutated in the consensus sequences to prevent binding to p53 (top) and pRb (bottom), and the amino acids mutated in the consensus sequences to prevent nuclear localization, respectively.

FIGS. 2A and 2B are schematic diagrams of Ag7.0 (SEQ ID NO: 13) (comprising the consensus E6-E7 fusion proteins derived from HPV16, HPV18, HPV52, and HPV58 separated by T2A peptides) and Ag7.1 (SEQ ID NO. 14) (comprising the mE6-mE7 fusion proteins derived from HPV16, HPV18, HPV52, and HPV58 separated by T2A peptides).

FIGS. 2C and 2D are graphs and blots showing results of experiments analyzing the expression of Ag7.0 and Ag7.1 in mouse cells lines by RT-qPCR and Western blot, respectively.

FIGS. 2E and 2F are graphs showing results of experiments analyzing the expression of Ag7.0 and Ag7.1 by flow cytometry.

FIGS. 3A and 3B are graphs showing results of experiments comparing the tumorigenicity of wild-type NIH/3T3 cells, NIH/3T3-Ag7.0 cells, NIH/3T3-Ag7.1 cells, the HeLa tumor cell line, and the TC-1 tumor cell line.

FIG. 3C is a graph showing safety of mE6-mE7 protein, as demonstrated by tumor volume of SCID mice, which were subcutaneously inoculated with NIH/3T3-mE6-mE7, NIH/3T3-wtE6-wtE7, and NIH/3T3, wherein each dot represents a mean value of tumor volume on indicated days; n=8.

FIG. 4A is a graph showing the binding capacity of tHA protein to CD209 in different concentrations as detected by ELISA assay.

FIG. 4B is a graph showing fusion structures of mE7-mE6, tHA-mE7-mE6 (GS), tHA-mE7-mE6 (H4), tHA-mE7-mE6 (2×H4), tHA-smnE7-smnE6 (GS) and tHA-smnE7-smnE6 (H5).

FIGS. 4C and 4D are blots and graphs showing results of experiments analyzing expression levels (by Western blot) and secretion (by ELISA) of HA-mE7-mE6 fusion proteins, respectively.

FIGS. 4E and 4F are graphs showing results of experiments analyzing the activation level of dendritic cells co-cultured with HA protein indicated by flow cytometric quantification of mCD80+ and mCD86+, respectively.

FIGS. 5A and 5B are graphs showing results of experiments quantifying T cell immunity induced by the tHA-mE7-mE6 mRNA vaccine by detecting total IgG against HPV antigen E6(16) and E7(16) respectively by ELISA; ***, P<0.001.

FIG. 5C is a graph showing results of flow cytometric analysis of mCD8⁺ splenocytes extracted from mice inoculated either subcutaneously or intramuscularly and co-cultured with a pool of E7(16) peptides, respectively.

FIG. 6A is a graph showing timeline of an experiment design to compare therapeutic efficacy of mE7-mE6-LNP and tHA-mE7-mE6-LNP; 5×10⁶ cells/dose, n=6.

FIGS. 6B, 6C and 6D are graphs showing tumor volume changes, body weight changing rate, and probability of survival, respectively, of mice in indicated groups; *, P<0.05.

FIG. 6E is a graph showing time line of an experiment design to test therapeutic efficacy of tHA-mE7-mE6-LNP in a low-dose TC1 model. 2×10⁴ cells/dose, n=15.

FIGS. 6F, 6G and 6H are graphs showing tumor volumes, body weight change and survival curves, respectively, of indicated time points; ***, P<0.001.

FIG. 7A is a graph showing design of re-challenge experiment; n=11.

FIG. 7B is a graph showing the volume curve of tumor in mice subjected to 1×10⁵ TC-1 re-challenge assay, wherein each point represents the tumor volume of each mouse.

FIG. 7C is a graph showing experiment design of preventing TC1 tumor formation by vaccination with tHA-mE7-mE6-LNP in naïve mice; n=15.

FIG. 7D is a graph showing the volume curve of tumor in mice in prophylactic assay, wherein each point represents the tumor volume of each mouse.

FIG. 7E is a graph showing change of mCD8⁺ cell population post tHA-mE7-mE6-LNP therapy.

FIG. 7F is a graph showing E7-TCR positive CD8⁺ cells observed in the spleens of mice post tHA-mE7-mE6-LNP therapy.

FIGS. 8A, 8B, 8C, 8D, 8E, and 8F are graphs showing the percentage of mIFN-γ⁺ cells, mCD69⁺ cells, mCD25⁺ cells, mCD107a⁺ cells, mGranzyme B⁺ cells, and mCD63⁺ cells in mCD8⁺ cells post tHA-mE7-mE6-LNP therapy; ***. P<0.001.

FIGS. 9A, 9B, 9C, 9D, 9E, 9F, 9G, and 9H shows immune cell infiltration and activation in the tumor post tHA-mE7-mE6-LNP therapy. FIG. 9A is a graph showing the percentage of mCD45⁺mCD3⁺ lymphocytes in total tumor tissue cells detected by flow cytometry. FIG. 9B is a graph showing the percentage of mCD8⁺ lymphocytes in mCD45⁺mCD3⁺ subset of tumor tissue cells. FIG. 9C is a graph showing the percentage of mTIM3⁺ cells in mCD8⁺ subset of tumor tissue cells. FIG. 9D is a graph showing the percentage of mCD69⁺mCD8⁺ cells in mCD3⁺ subset of tumor tissue cells. FIG. 9E is a graph showing the percentage of mCD8⁺ mIFN-γ⁺ lymphocytes after 6 hours of HPV16 E7 PepMix stimulation. FIG. 9F is a graph showing the percentage of mCD45⁺mCD11c⁺ subset in total tumor tissue cells. FIG. 9G is a graph showing the percentage of mCD45⁺mCD3-mNK1.1⁺ subset of tumor tissue cells. FIG. 9H is a graph showing the intensity of mCD80 in mCD11c⁺ subset of tumor tissue cells. Each dot represents data from one mouse; n=6; *, P<0.05; **, P<0.01; ***, P<0.001.

FIG. 10 is a graph showing the relative mRNA expression level of immunological genes in tumor post tHA-mE7-mE6-LNP therapy; n=6; *, P<0.05; **, P<0.01; ***, P<0.001.

Corresponding reference numerals indicate corresponding parts throughout the drawings.

### DETAILED DESCRIPTION OF THE INVENTION

### Definitions

As used herein “immunogenic” refers to a compound or compositions which causes or is capable of causing an immune response.

As used herein “expression cassette” refers to one or more gene(s) and the sequence(s) which control their expression. Typically, an expression cassette comprises a regulatory sequence and a nucleotide sequence to be expressed by the expression cassette. Within certain embodiments the expression cassette comprises a promoter sequence, a nucleic acid sequence, and in eukaryotes, a polyadenylation site.

As noted above, the present invention provides compositions and methods comprising one or more immunogenic proteins which have been derived from a strain of HPV which causes or is associated with causing cancer, which are mutated so that they do not cause increased cell proliferation. Within various embodiments of the invention the immunogenic protein is derived from an HPV strain which is associated with causing cancer or the proliferation of cells in subjects may be similarly mutated. Representative examples include, but are not limited to HPV strains 16, 18, 31, 33, 35, 39, 45, 51, 52, 56, 58, 59, 66 and 68. Within related embodiments of the invention the immunogenic protein further comprises a cell-targeting moiety such as the HA protein. Within various embodiments of the invention, the mutation is one or more amino acid mutations or changes (e.g., a substitution, insertion, and/or deletion (e.g., a truncation) which substantially maintains immunogenicity similar to that of the native protein, but which no longer causes cell proliferation.

Within further embodiments the immunogenic protein may be fused to a cell targeting moiety such as HA protein in order to increase its utility as a vaccine.

In order to further an understanding of the various embodiments herein, the following sections are provided to more specifically describe various embodiments of the invention: A. Immunogenic Proteins; B. Vaccines; C. Therapeutic Compositions; and D. Administration.

a. Immunogenic Proteins

As noted above, compositions and methods are provided that may be used to prevent, treat, or ameliorate the effects of HPV, comprising one or more immunogenic proteins.

More specifically, within one embodiment of the invention immunogenic proteins are provided from a strain of HPV which causes or is associated with causing cancer, which are mutated so that they do not cause increased cell proliferation. For example, as noted above the wild-type early protein 7 (E7) from HPV strain 16 (HPV16) and early protein 6 (E6) from HPV strain 16 (HPV16) [CN 101100672 B] are tumorigenic. They localize into the cell nucleus and interact with tumor suppressors, such as p53 and pRB, to cause aberrant cell proliferation. In preferred embodiments of the invention, amino acid residues on E6 and E7 derived from HPV strain 16 that are essential for interacting with p53 and pRB are mutated. As a result, the mutated E6 and E7 do not cause increased cell proliferation.

Nuclear localized E7 and E6 are presented by MHC-I at a low level, causing E7/E6-positive cells to be less visible to the immune system. Antigen presentation involves proteasome-dependent peptide generation in the cytoplasm, TAP-dependent peptide transport into the lumen of the endoplasmic reticulum (ER) where binding to MHC-I molecules occurs, and transit of stable peptide-loaded MHC-I molecules through the secretory pathway to the plasma membrane for T cell recognition. To prevent mutated E7 (mE7) and mutated E6 (mE6) from entering the nucleus, the essential NLS (nuclear localization signal) of mE7 and mE6 are modified to prevent nuclear localization. The resulting non-nuclear localized smnE7-smnE6 induces stronger CD8⁺ T cell responses than the nuclear-localized protein.

In another embodiment, amino acid residues that are responsible for interacting with p53 and/or pRB (e.g., E6 and E7) from other HPV strains such as HPV strains 18, 31, 33, 35, 39, 45, 51, 52, 56, 58, 59, 66 and 68, can similarly be mutated to prevent cell proliferation, and utilized in the compositions and methods provided herein. Within related embodiments of the invention the immunogenic protein further comprises a cell-targeting moiety such as the HA protein. Within various embodiments of the invention, the mutation is one or more amino acid mutations or changes (e.g., a substitution, insertion, and/or deletion (e.g., a truncation)) which substantially maintains immunogenicity similar to that of the native protein, but which no longer causes cell proliferation.

The mutated immunogenic HPV proteins provided herein (such as E7-E6) can be further modified by fusing it with a cell targeting moiety such as hemagglutinin A protein (also referred to as “HA protein”) from influenza virus A (“IVA”). Briefly, HA protein interacts with DC-specific ICAM-grabbing non-integrin (DC-SIGN/CD209 protein), which is widely expressed on the surface of iDC (immature dendritic cells) and macrophages. HA protein guides the mutated E7-E6 antigen towards DC and macrophages by binding to DC-SIGN/CD209. Upon entering the iDCs, HA protein promotes the activation and maturation of iDCs, leading to more efficient antigen presentation through APCs (antigen presenting cells). Within certain embodiments of the invention, HA protein is derived from influenza A (IVA) subtypes H1, H2, H3, H4, H5, H6, H7, H8, H9, H10, H11, H12, H13, H14, H15, H16, H17, or H18. As used within the context of the present invention, “HA protein” refers to both the entire protein sequence, as well as peptide components thereof that are responsible for binding of the influenza virus to the cell that is being infected. In other embodiments, the mutated immunogenic HPV proteins provided herein (such as E7-E6) can be fused with a different cell targeting moiety to guide the mutated HPV antigen(s) towards dendritic cells and macrophages.

Within certain embodiments of the invention, the mutated immunogenic HPV proteins provided herein are fused to truncated HA (tHA). In tHA the transmembrane and intracellular domains are deleted to allow efficient secretion and spread of protein. Within preferred embodiments of the invention, tHA fused with mE7-mE6 allows highly efficient uptake of antigen by APC and high therapeutic efficacy.

Representative and non-exhaustive examples of additional suitable cell targeting moieties include: (a) Ebola virus glycoprotein (see, e.g., Alvarez, C. P. et al., C-type lectins DC-SIGN and L-SIGN mediate cellular entry by Ebola virus in cis and in trans. J. Virol., 2002. 76(13): p. 6841-4); (b) envelope glycoprotein gp120 of HIV (see e.g., Becer, C. R., et al., High-affinity glycopolymer binding to human DC-SIGN and disruption of DC-SIGN interactions with H/V envelope glycoprotein. Journal of the American Chemical Society, 2010. 132(43): p. 15130-15132); (c) Marburg virus glycoprotein (see e.g., Marzi, A., et al., DC-SIGN and DC-SIGNR interact with the glycoprotein of Marburg virus and the S protein of severe acute respiratory syndrome coronavirus. J Virol, 2004. 78(21): p. 12090-5); and (d) E2 glycoprotein from hepatitis C virus (see e.g., Pöhlmann, S., et al., Hepatitis C virus glycoproteins interact with DC-SIGN and DC-SIGNR. Journal of virology, 2003. 77(7): p. 4070-4080); all of which are incorporated by reference in their entirety.

The immunogenic proteins (and/or cell targeting moiety), can readily be made by commercially available synthetic means (see, e.g., commercial manufacturers such as Profacgen, Shirley, NY and GenScript, Piscataway NJ), or by cloning, expression and purification of a recombinant sequence which encodes the protein (see generally, Textbook on Cloning, Expression and Purification of Recombinant Proteins 1st ed. 2022 Edition by Kakoli Bose (Editor); ISBN-10 9811649863; ISBN-13 978-9811649868).

Immunogenicity of the proteins can also be assessed by readily available assays (see, e.g., Stanley, “Immunobiology of HPV and HPV vaccines, Gynecologic Oncology, Vol. 109, Issue 2, Supplement, May 2008, Pages S15-S21; Michel et al., “Enhanced Immunogenicity of HPV 16 E7 Fusion Protein in DNA Vaccination, Virology, Vol. 294, Issue 1, 1 Mar. 2002, Pages 47-59; Turner and Hug, “HPV vaccines: Translating immunogenicity into efficacy”, Human Vaccines & Immunotherapeutics, Vol. 12, 2016, Pages 1403-1405, https://doi.org/10.1080/21645515.2015.1103936; and Kim et al., “Purification and immunogenicity study of human papillomavirus type 16L1 protein in Saccharomyces cerevisiae”, Journal of Virological Methods, Vol. 139, Issue 1, January 2007, Pages 24-30; “T-cell dependent immunogenicity of protein therapeutics: Preclinical assessment and mitigation”, Clinical Immunology, Vol. 149, Issue 3, Part B, December 2013, Pages 534-555, http://dx.doi.org/10.1016/j.clim.2013.09.006, all of which are incorporated by reference in their entirety.

### B. Vaccines

Therapeutic compositions are provided that may be used to prevent, treat, or ameliorate the effects of HPV. Briefly, the vaccine may be formulated as a protein/peptide, or, as a genetic vaccine (see generally “Vaccine Development and Manufacturing” (Wiley Series in Biotechnology and Bioengineering Book 5) 1st Edition, Kindle Edition by Emily P. Wen (Editor), Narahari S. Pujar (Editor), Ronald Ellis (Editor); ISBN-13 978-0470261941). Representative examples of genetic vaccines include viral vectors, FNA vaccines and DNA vaccines. Within one embodiment of the invention, the genetic vaccine is a viral vector (e.g., derived from an adenovirus, coxsackievirus, H-1 parvovirus, herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picornavirus such as poliovirus or Seneca valley virus, pox viruses such as vaccinia virus (e.g. Copenhagen, Indiana Western Reserve, and Wyeth strains), reovirus, or retrovirus such as murine leukemia virus. Within certain embodiments the viral vector is an oncolytic viral vector. Representative examples of viral vectors are described in: U.S. Pat. Nos. 8,147,822 and 9,045,729 (oncolytic rhabdovirus/VSV); U.S. Pat. No. 9,272,008 (oncolytic Measles virus); U.S. Pat. Nos. 7,223,593, 7,537,924, 7,063,835, 7,063,851, 7,118,755, 8,216,564, 8,277,818, and 8,680,068 (oncolytic herpes virus vectors); and U.S. Pat. No. 8,980,246 (oncolytic vaccinia virus), all of which are incorporated by reference in their entirety.

Within particularly preferred embodiments of the invention, the vaccine is an mRNA-based vaccine, including but not limited to non-replicable mRNA and self-amplifiable mRNA. Briefly, mRNA vaccines use a copy of messenger RNA to introduce RNA into a cell, and thereby to elicit an immune response. Within further embodiments, in order to improve delivery efficiency in vivo, formulation methods (e.g., liposomes and nanoparticles such as lipid nanoparticles “LNP”) can be used to encapsulate mRNA. Within preferred embodiments the mRNA may be encapsulated into a liposome, or a nanoparticle based delivery system.

Representative examples of methods for making RNA-based vaccines (and RNA-based vaccine delivery systems) are described in “RNA Vaccines: Methods and Protocols” (Methods in Molecular Biology, 1499) 1st ed. 2017 Edition by Thomas Kramps (Editor), Knut Elbers (Editor), ISBN-10 1493964798; ISBN-13 978-1493964796; “Vaccine Adjuvants: Methods and Protocols” (Methods in Molecular Biology, 1494) 1st ed. 2017 Edition by Christopher B. Fox (Editor); ISBN-10 1493964437; ISBN-13 978-1493964437; Kowalski P S, Rudra A, Miao L, Anderson D G (April 2019). “Delivering the Messenger: Advances in Technologies for Therapeutic mRNA Delivery”. Mol Ther. 27 (4): 710-28. doi:10.1016/j.ymthe.2019.02.012. PMC 6453548. PMID 30846391; Verbeke R, Lentacker I, De Smedt S C, Dewitte H (October 2019). “Three decades of messenger RNA vaccine development”. Nano Today. 28: 100766. doi:10.1016/j.nantod.2019.100766. hdl:1854/LU-8628303. S2CID 202221207; Pardi N, Hogan M J, Porter F W, Weissman D (April 2018). “mRNA vaccines—a new era in vaccinology”. Nature Reviews Drug Discovery. 17 (4): 261-279. doi:10.1038/nrd.2017.243. ISSN 1474-1784. PMC 5906799. PMID 29326426; and the following patents and patent applications: U.S. Ser. No. 10/207,009B2 entitled “Method for cellular RNA expression”; PCT/EP2010/004760 Entitled “Vaccine composition comprising 5′-cap modified RNA”; U.S. Ser. No. 10/485,884B2 entitled “RNA formulation for Immunotherapy”; U.S. Ser. No. 11/020,477B2 entitled “RNA vaccines”; and U.S. Ser. No. 10/106,800B2 entitled “Modification of RNA, producing an increased transcript stability and translation efficiency”, U.S. Ser. No. 10/106,490 entitled “Lipids and lipid nanoparticle formulations for delivery of nucleic acids””; US 2019/0274968 entitled “Nucleoside modified RNA for inducing an adaptive immune response”; US 2016/0317676 entitled “Methods and compositions for delivery of nucleic acids”; AU 2018/256877 entitled “Novel carbonyl lipids and lipid nanoparticle formulations for delivery of nucleic acids”; U.S. Pat. No. 11,357,856 entitled “Lipids for delivery of active agents”; all of which are incorporated by reference in their entirety.

### C. Therapeutic Compositions

The present invention also provides therapeutic compositions comprising the above-noted immunogenic proteins (and/or nucleic acid sequences such as mRNA encoding such proteins), as well as pharmaceutical compositions.

In certain embodiments, the compositions will further comprise a pharmaceutically acceptable carrier. The phrase “pharmaceutically acceptable carrier” is meant to encompass any carrier, diluent or excipient that does not interfere with the effectiveness of the immunogenic protein (and/or nucleic acid sequences such as mRNA encoding such proteins) and that is not toxic to the subject to whom it is administered (see generally Remington: The Science and Practice of Pharmacy, Lippincott Williams & Wilkins; 21st ed. (May 1, 2005 and in The United States Pharmacopoeia: The National Formulary (USP 40—NF 35 and Supplements). Within certain embodiments of the invention, the pharmaceutical composition comprises a liposome or nanoparticle (e.g., LNP) that carries and/or facilitates the delivery of an RNA-based vaccine.

In the case of immunogenic proteins as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the recombinant oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the vaccine and will preferably not induce an immune response in the individual (subject) receiving the composition (and will preferably be administered without undue toxicity).

The compositions provided herein can be provided at a variety of concentrations. For example, the compositions may be administered as a single dose or as multiple doses spread out over time. Doses may be administered daily, weekly, biweekly, monthly, or bimonthly, and dosage frequency may be cyclical, with each cycle comprising a repeating dosage pattern (e. g. once a week or biweekly dose administration for about 4 weeks comprising one cycle, repeating for up to about 24 cycles).

The compositions may be stored at a temperature conducive to stable shelf-life and includes room temperature (about 20° C.), 4° C., −20° C., −80° C., and in liquid N₂. Because compositions intended for use in vivo generally do not have preservatives, storage will generally be at colder temperatures. Compositions may be stored dry (e.g., lyophilized) or in liquid form.

### D. Administration

In addition to the compositions described herein, various methods of using such compositions to prevent, treat and/or ameliorate HPV-related disease, comprising the step of administering to a subject an effective dose or amount of an immunogenic protein as described herein to a subject.

The terms “effective dose” and “effective amount” refers to amounts of the immunogenic protein that is sufficient to effect treatment of a subject.

The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

The term “prevent” or “preventing” means to lessen the incidence, occurrence, or, likelihood of occurrence of a HPV-related disease.

The term “HPV-related disease” incudes both skin or mucous membrane growths (e.g., warts), as well as different types of cancers or precancerous conditions. Within certain embodiments of the invention, the HPV-related disease is HPV-positive cancer. “HPV-positive cancer” as used herein refers to a cancer that is positive for HPV. In certain embodiments, an HPV positive cancer tumor expresses one or more proteins derived from an HPV or has a nucleotide sequence encoding the one or more proteins derived from an HPV. Examples of cancer include, but are not limited to adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor.

Precancerous conditions are known in the art as a group of disorders that have a malignant predisposition, which are conditions associated with a risk of turning into a particular type of cancer. A precancerous condition may be, for example, a genetic predisposition for the cancer, cellular or morphological changes in a tissue, also known as a “lesion”, indicative of a precancerous condition, or a gene mutation or mutations associated with increased risk for a particular type of cancer. Representative examples of precancerous conditions that may be treated in accordance with the present invention include cervical dysplasia or squamous intraepithelial lesion (e.g., diagnosed by pap smear), prostatic intraepithelial neoplasia (PIN); superficial bladder cancer, also known as transitional cell carcinoma in situ, precancerous lesions of the breast, precancerous lesions of the lung, actinic keratosis, Barrett's esophagus; precancerous melanoma moles, precancerous conditions of the uterus/vulva, precancerous conditions of the ovary, atrophic gastritis, precancerous conditions of the oral cavity, general dysplastic conditions, squamous metaplasia, intraepithelial neoplasia, precancerous conditions in the head or neck, familial adenomatous polyposis (Gardner's syndrome), sporadic adenomatous polyposis, hereditary non-polyposis colon cancer (Lynch syndrome), and Crohn's disease.

In the context of HPV-related cancer, the term “treat” or “treating” refers to amounts of immunogenic protein (and/or nucleic acid sequence encoding such protein) that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The optimal or appropriate dosage regimen of the immunogenic protein is readily determinable within the skill of the art, by the attending physician based on patient data, patient observations, and various clinical factors, including for example a subject's size, body surface area, age, gender, and the particular recombinant oncolytic virus being administered, the time and route of administration, the type of cancer being treated, the general health of the patient, and other drug therapies to which the patient is being subjected.

Immunogenic proteins (and/or nucleic acid sequences such as mRNA which encode such proteins) may be formulated as medicaments and pharmaceutical compositions for clinical use and may be combined with a pharmaceutically acceptable carrier, diluent, excipient or adjuvant. The formulation will depend, at least in part, on the route of administration. Suitable formulations can be fluid, gel, paste or solid forms, although, lipid-based nanoparticles are particularly preferred for mRNA-based vaccines. Formulations may be provided to a subject or medical professional.

The following are some exemplary numbered embodiments of the present disclosure.
1. An immunogenic protein, comprising an amino acid sequence derived from one or more proteins which are responsible for cell proliferation, and wherein said one or more proteins are derived from an HPV strain associated with causing cancer.
2. The protein of embodiment 1, wherein said HPV strain associated with causing cancer is selected from the group consisting of HPV strain 16, 18, 31, 33, 35, 39, 45, 51, 52, 56, 58, 59, 66 or 68.
3. The protein of embodiment 1 or 2, wherein said protein is mutated such that it does not cause cell proliferation.
4. The protein according to embodiment 3 wherein said mutation is one or more amino acid changes selected from the group consisting of substitutions, insertions and deletions.
5. The protein according to embodiment 3 wherein said mutation eliminates or diminishes interactions with p53/pRB.
6. The protein according to embodiment 3 wherein said mutation reduces or prevents nuclear localization.
7. The protein according to embodiment 3 wherein said mutation induces a stronger immune response, and preferably, a stronger T cell response. Within other embodiments the mutation improves safety (e.g., makes the protein less tumorigenic) and, within preferred embodiments, does not substantively decrease the immune response as compared to unmutated E6 and E7.
8. The protein according to embodiment 3 wherein said mutated protein is E6 or E7.
9. An immunogenic fusion protein, comprising a protein according to any one of embodiments 1 to 8, and a cell-targeting moiety. Within other embodiments the fusion protein increases tumor lysis.
10. The immunogenic fusion protein according to embodiment 9, wherein said cell targeting moiety is all or a portion of an HA protein (e.g., a truncated HA protein).
11. The immunogenic fusion protein according to embodiment 9 wherein said cell-targeting moiety is selected from the group consisting of Ebola virus glycoprotein, envelope glycoprotein gp120 of HIV, Marburg virus glycoprotein, E2 glycoprotein from hepatitis C virus, and the spike protein of COVID19.
12. An immunogenic fusion protein comprising:
a first amino acid sequence having at least 80%, 85%, 90%, or, 95% sequence identity sequence identity with SEQ ID NO:3 (mE6(16)), and
a second amino acid sequence having at least 80%, 85%, 90%, or, 95% sequence identity sequence identity with SEQ ID NO:4 (mE7(16)).
13. An immunogenic fusion protein comprising:
a first amino acid sequence having at least 80%, 85%, 90%, or, 95% sequence identity sequence identity with SEQ ID NO:5 (smnE6(16)), and
a second amino acid sequence having at least 80%, 85%, 90%, or, 95% sequence identity sequence identity with SEQ ID NO:6 (smnE7(16)).
14. An immunogenic fusion protein, comprising a sequence according to SEQ ID. NO. 7, or, a sequence with at least 80%, 85%, 90%, or, 95% identity thereto.
15. An immunogenic fusion protein, comprising a sequence according to SEQ ID. NO. 8 or, a sequence with at least 80%, 85%, 90%, or, 95% identity thereto.
16. An immunogenic fusion protein, comprising a sequence according to SEQ ID. NO. 9, or, a sequence with at least 80%, 85%, 90%, or, 95% identity thereto.
17. An immunogenic fusion protein, comprising a sequence according to SEQ ID. NO. 10, or, a sequence with at least 80%, 85%, 90%, or, 95% identity thereto.
18. An immunogenic fusion protein, comprising a sequence according to SEQ ID. NO. 11, or, a sequence with at least 80%, 85%, 90%, or, 95% identity thereto.
19. An immunogenic fusion protein, comprising a sequence according to SEQ ID. NO. 12, or, a sequence with at least 80%, 85%, 90%, or, 95% identity thereto.
20. An immunogenic fusion protein, comprising a sequence according to SEQ ID. NO. 13, or, a sequence with at least 80%, 85%, 90%, or, 95% identity thereto.
21. An immunogenic fusion protein, comprising a sequence according to SEQ ID. NO. 14, or, a sequence with at least 80%, 85%, 90%, or, 95% identity thereto.
22. A nucleic acid sequence which encodes an immunogenic protein or immunogenic fusion protein according to any one of embodiments 1 to 21. Within certain embodiments of the invention the nucleic acid sequence is an isolated nucleic acid sequence.
23. The nucleic acid sequence according to embodiment 22 wherein said nucleic acid sequence is an mRNA sequence.
24. An expression cassette which directs the expression of an immunogenic protein or immunogenic fusion protein according to any one of embodiments 1 to 21.
25. A composition comprising an mRNA sequence encoding an immunogenic protein or immunogenic fusion protein according to any of embodiments 1 to 21.
26. The composition according to embodiment 25, wherein said composition is contained within a nanoparticle or liposome.
27. A pharmaceutical composition, comprising an immunogenic protein or immunogenic fusion protein according to any one of embodiments 1 to 21.
28. A pharmaceutical composition comprising an isolated nucleic acid sequence according to embodiment 22 or 23. Within preferred embodiments the isolated nucleic acid sequence is formulated with an nanoparticle or liposome.
29. A pharmaceutical composition comprising an expression cassette according to embodiment 24.
30. A pharmaceutical composition comprising an mRNA sequence according to embodiment 25 or embodiment 26.
31. A vaccine which induces an immune response in a subject, comprising a pharmaceutically acceptable vehicle and a pharmaceutically effective amount of an immunogenic protein or immunogenic fusion protein according to any one of embodiments 1 to 21.
32. A vaccine which induces an immune response in a subject, comprising a pharmaceutical composition according to embodiment 30.
33. A method of treating an HPV-related disease in a subject in need thereof, comprising administering a therapeutically effective amount of a vaccine according to embodiments 31 or 32, to a subject.
34. The method according to embodiment 33, wherein the HPV-related disease is cancer.
35. The method according to embodiment 34, wherein the HPV-related disease is HPV-positive cancer.
36. The method according to embodiment 35, wherein the cancer comprises adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor.
37. The method according to embodiment 33, wherein the HPV-related disease is a precancerous condition, or, recurrent respiratory papillomatosis (RRP).
38. The method according to embodiment 37, wherein the precancerous condition comprises cervical dysplasia or squamous intraepithelial lesion, prostatic intraepithelial neoplasia, superficial bladder cancer, precancerous lesions of the breast, precancerous lesions of the lung, actinic keratosis, Barrett's esophagus, precancerous melanoma moles, precancerous conditions of the uterus/vulva, precancerous conditions of the ovary, atrophic gastritis, precancerous conditions of the oral cavity, general dysplastic conditions, squamous metaplasia, intraepithelial neoplasia, precancerous conditions in the head or neck, familial adenomatous polyposis (Gardner's syndrome), sporadic adenomatous polyposis, hereditary non-polyposis colon cancer (Lynch syndrome), and Crohn's disease.
39. A method of treating HPV in a subject in need thereof, comprising administering a therapeutically effective amount of a vaccine according to embodiments 31 or 32, to a subject.
40. The method according to any one of embodiments 33-39, wherein the vaccine is administered to the subject intratumorally, subcutaneously, intramuscularly, intradermally or intravenously.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y,” and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

“Isolated” nucleic acid molecule means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. Applicants reserve the right to physically incorporate into this specification any and all materials and information from any such patents, publications, scientific articles, web sites, electronically available information, and other referenced materials or documents.

The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The Examples and preparations provided below further illustrate and exemplify the compounds of the present invention and methods of preparing such compounds. It is to be understood that the scope of the present invention is not limited in any way by the scope of the following Examples and preparations. In the following Examples, molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art. The starting materials and various reactants utilized or referenced in the examples may be obtained from commercial sources, or are readily prepared from commercially available organic compounds, using methods well-known to one skilled in the art.

### EXAMPLES

### Example 1

### Generating Consensus Sequences and Mutating Portions of E6 and E7 Proteins

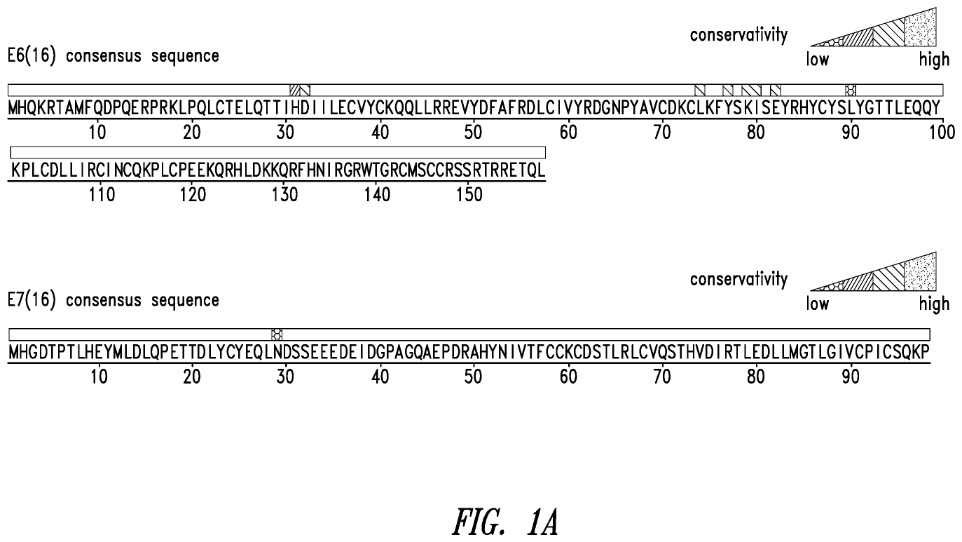

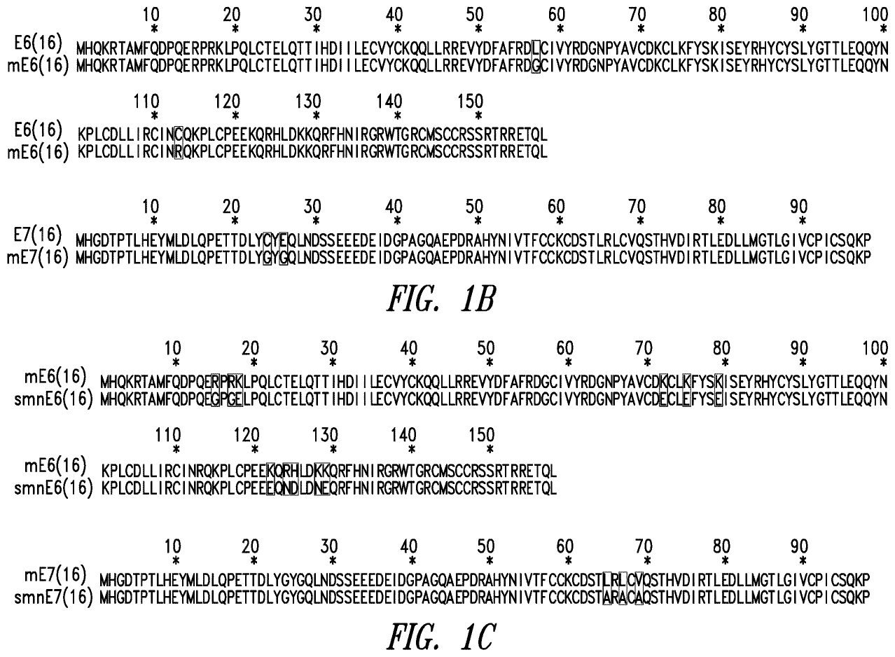

The consensus sequence of E6 (HPV strain 16) and E7 (HPV strain 16), also referred to as E6 (16) (SEQ ID NO:1) and E7 (16) (SEQ ID NO:2), respectively, were generated by aligning all NCBI published (as of 2021.12.31) wild-type E6 (16) and E7 (16) amino acid sequences that were identified in China. The E6(16) and E7(16) consensus sequences are set forth in FIG. 1A. The essential residues within the P53-binding motif of E6 (16) were mutated, resulting in mE6 (16) (SEQ ID NO:3). Likewise, the essential residues within the pRB-binding motif of E7 (16) were mutated to create mE7 (16) (SEQ ID NO:4). These residues are highlighted in the amino acid sequences set forth in FIG. 1B. The essential residues within nuclear localization signal (NLS) motifs of mE6 (16) and mE7 (16) were mutated to block the NLS function. The mutants were named smnE6 (16) (SEQ ID NO:5) and smnE7 (16) (SEQ ID NO:6), respectively. The amino acid sequences of these mutants are set forth in FIG. 1C.

### Example 2

Safety Evaluation of mE6-mE7 Fusion Proteins

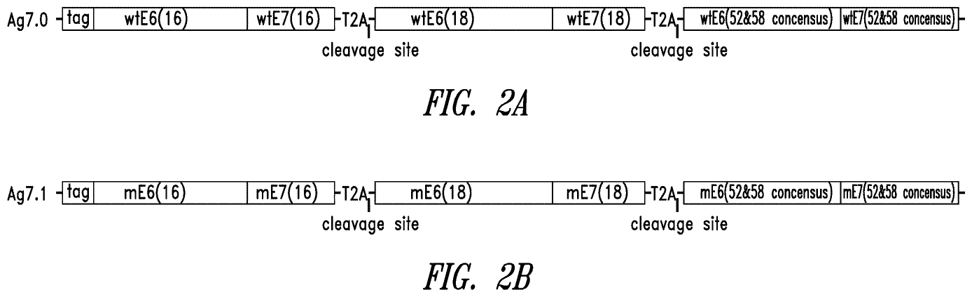

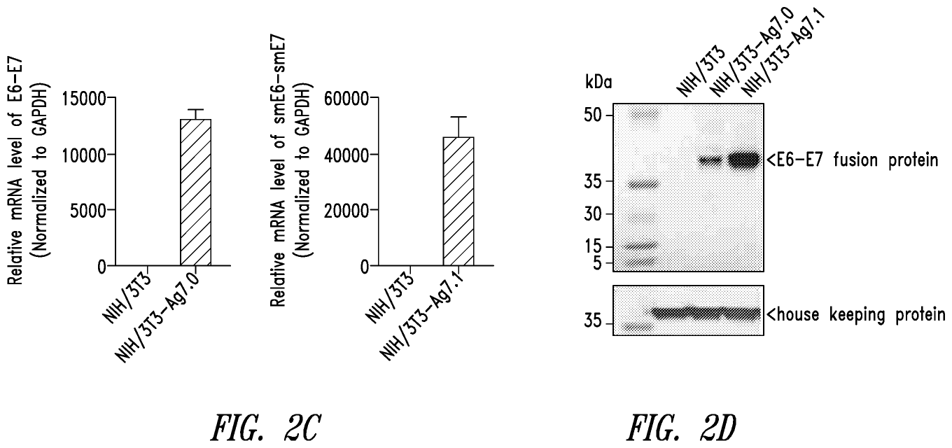

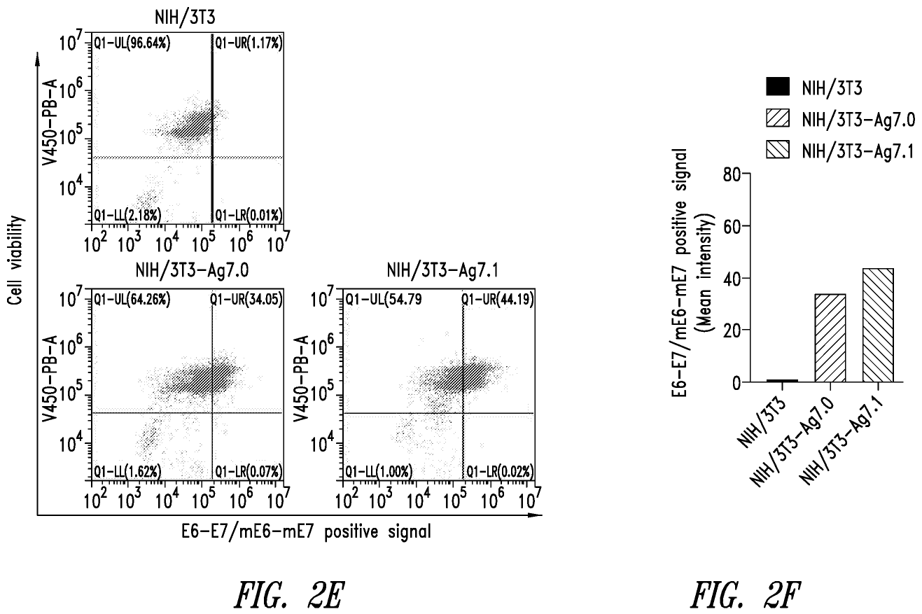

To test the safety of mE6 and mE7, the non-tumorigenic mouse cell line NIH/3T3 was used to stably express proteins Ag7.0 (SEQ ID NO. 13) and Ag7.1 (SEQ ID NO. 14). The protein Ag7.0 comprised the consensus E6-E7 fusion protein sequences from HPV16, HPV18, HPV52, and HPV58, with fusion proteins derived from different strains separated by a “self-cleaving” T2A peptide as depicted in FIG. 2A. The protein Ag7.1 comprised the mE6-mE7 fusion protein sequences from HPV16, HPV18, HPV52, and HPV58, with fusion proteins derived from different strains separated by a “self-cleaving” T2A peptide as shown in FIG. 2B. A protein tag was fused to the N-terminus of Ag7.0 and Ag7.1 to aid in detecting protein expression. A lentiviral vector system was used to generate the above cell lines. The cell lines were then cultured in medium with low melting point agarose to promote cell colony formation. The mRNA levels of Ag7.0 and Ag7.1 genes were analyzed using RT-qPCR, as shown in the graph presented in FIG. 2C. Western blotting was used to identify the levels of Ag7.0 and Ag7.1 protein, as shown in the blot presented in FIG. 2D. The Ag7.0- and Ag7.1-expressing cell populations were quantified using flow cytometry by labeling Ag7.0 and Ag7.1 with fluorophore-tagged antibodies, as shown in the graphs presented in FIGS. 2E and 2F. The results confirmed that the novel generated cell lines NIH/3T3-Ag7.0 and NIH/3T3-Ag7.1 express the genes of interest.

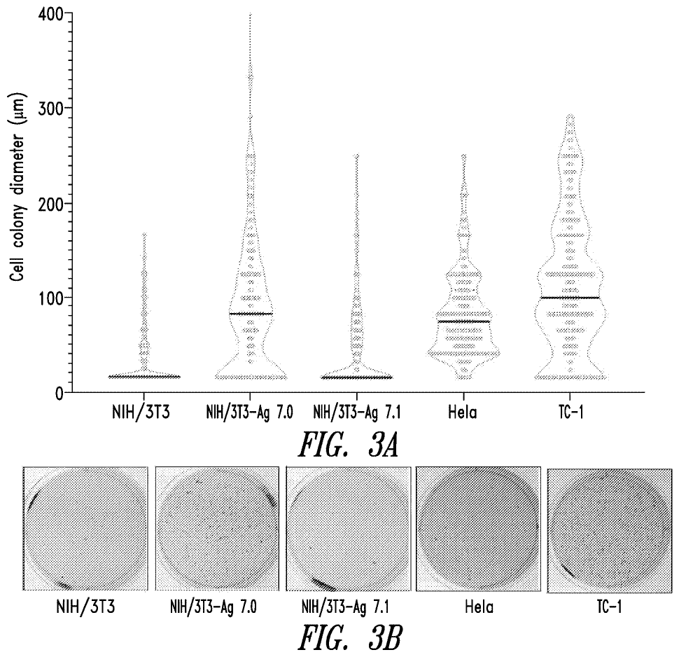

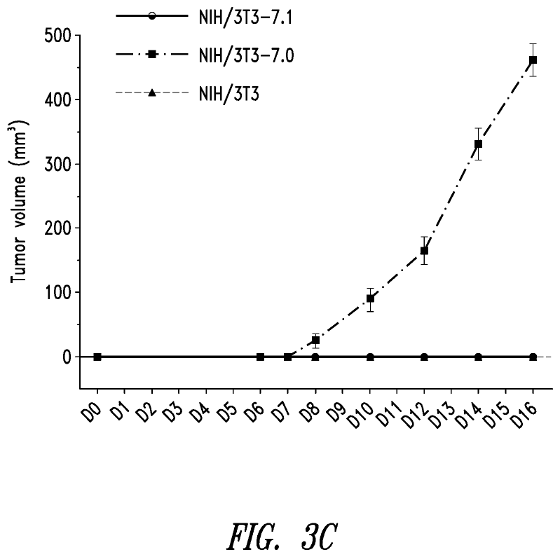

Tumorigenicity was tested by observing the ability of a single cell to grow into a colony in soft agarose-containing medium as shown in the graph and photographs presented in FIGS. 3A and 3B. Wild-type NIH/3T3 cells [NIH/3T3(wt)] were used as a negative control. HeLa and TC-1 tumor cell lines were used as positive controls to represent the colony formation of cancer cells. Results showed that NIH/3T3-Ag7.0 forms fast-growing colonies with an average diameter comparable to colonies derived from HeLa and TC-1 cells. The average diameter of cell colonies derived from NIH/3T3-Ag7.1 and NIH/3T3(wt) cells were close to the diameter of a single cell, indicating that Ag7.1 did not cause cell transformation. Overall, the results indicated that mutating the p53/pRB-interacting motifs in E6/E7 can successfully abolish the tumorigenicity of E6/E7 proteins from four different HPV strains, suggesting that similar E6/E7 modifications may be used to the same effect across a broad range of HPV strains. FIG. 3C demonstrates safety of tHA-mE7-mE6-LNP.

### Example 3

Fusion of mE7-mE6 with HA Promotes Secretion of the Fusion Protein and Enhances iDC Activation

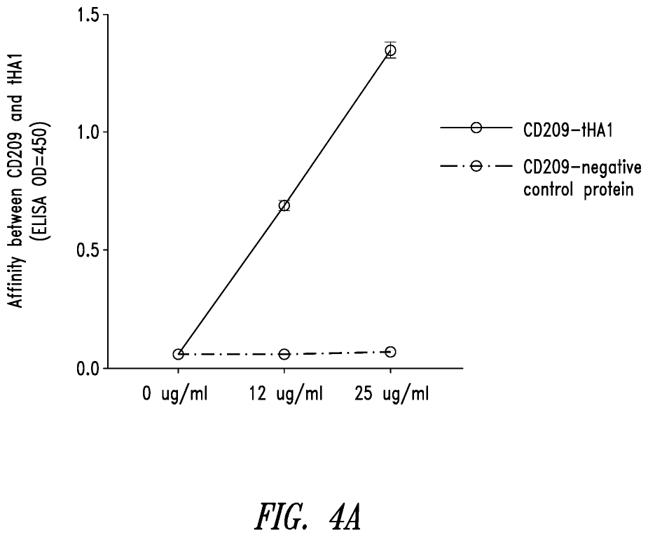

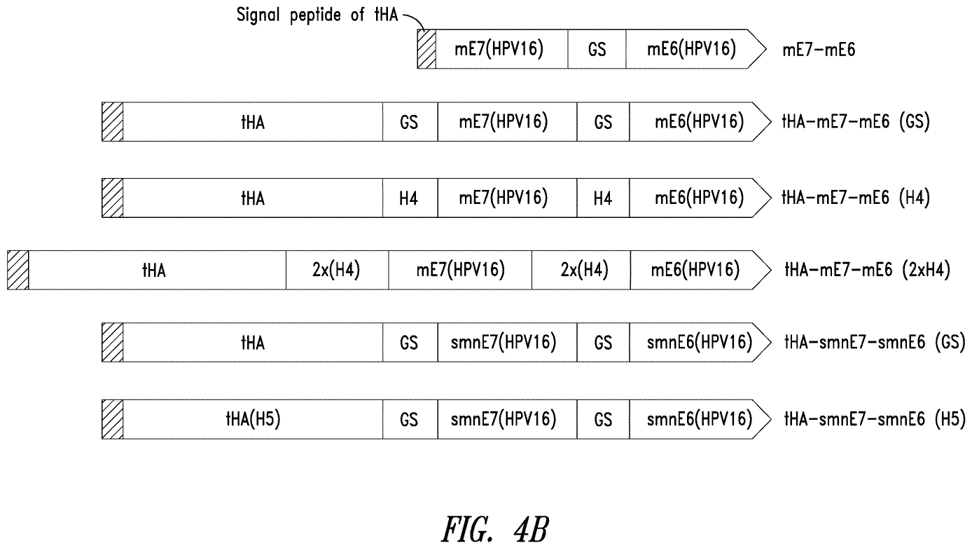

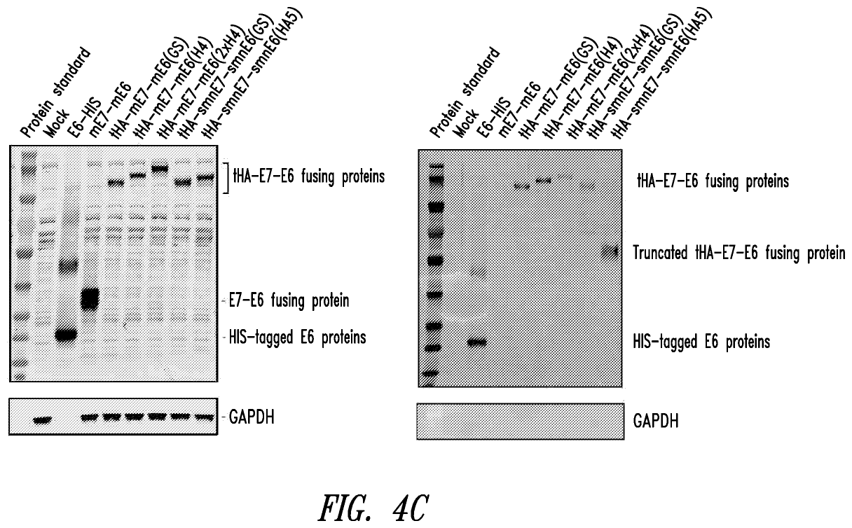

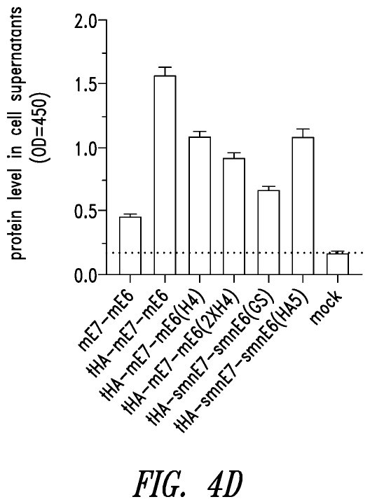

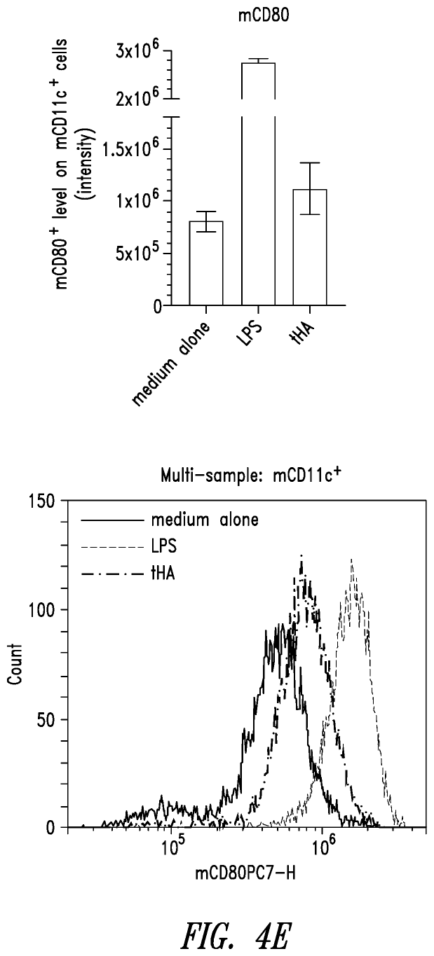

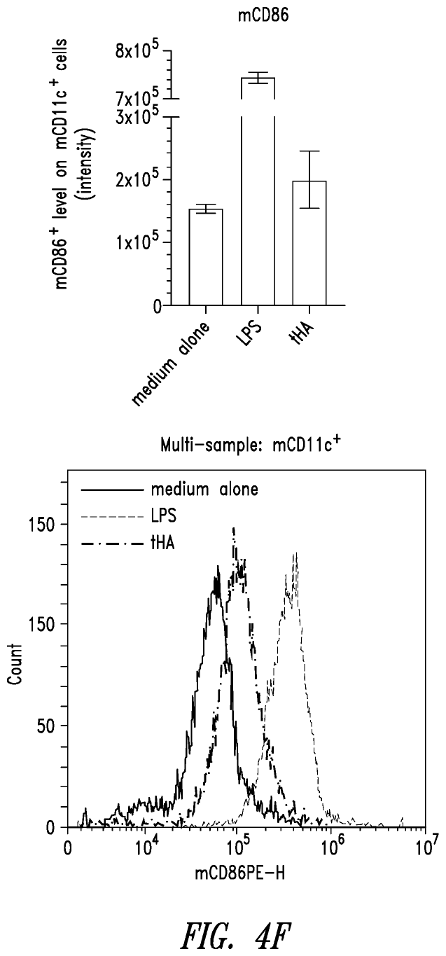

To test the secretion of HA-mE7-mE6 fusion proteins and their impact on iDC activation, several combinations of fusion proteins were generated and tested. The transmembrane and intracellular domains of HA were removed to allow the protein to be secreted. HA initiates IVA infection by interacting with CD209, a C-type lectin receptor expressed in DCs. An ELISA assay was performed to test whether deleting the transmembrane and intracellular domains of HA affected the interaction with CD209, and E6-HIS protein was used as control. The results indicated that tHA effectively associated with CD209, as shown in the graph presented in FIG. 4A. The tHA signals were intensified with increased protein concentration of CD209 coated on the plate surface.

Briefly, cell lines were transfected with in vitro transcribed products (IVT mRNA) listed in Table 1, wherein the fusion structures of mE7-mE6, tHA-mE7-mE6 (GS), tHA-mE7-mE6 (H4), and tHA-mE7-mE6 (2×H4) are demonstrated in FIG. 4B. Cell lysates and cell supernatants were collected for Western blotting or ELISA analysis to detect protein expression in cells and secretion into the extracellular environment. All tested proteins were expressed with their expected molecular weight as indicated by Western blot presented in FIG. 4C. ELISA results showed the fusion proteins with HA subtypes H1 or H5 were successfully secreted into the cell culture medium, as shown in the graph presented in FIG. 4D. Overall, the data indicated that H1-mE7-mE6 fusion proteins were stably expressed with the tested linkers and were successfully secreted into the cytoplasm.

An ex vivo iDC activation assay was used to test activation induced by the HA protein and by the HA-mE7-mE6 fusion protein. Bone marrow cells were isolated from healthy C57BL6 mice (6-8-week-old females) and cultured with mGM-CSF (murine GM-CSF) protein. After a period of differentiation, cells were then co-cultured with HA protein or HA-mE7-mE6 fusion protein for 1 day. The cells were harvested for flow cytometry analysis, wherein the mCD11c⁺ marker was used to indicate the total DC population. RPMI-1640 medium with and without LPS (lipopolysaccharide) were used as positive and negative controls. mCD80⁺ and mCD86⁺ were used to indicate the activation level of DC cells, as shown in the graphs presented in FIGS. 4E and 4F. The results indicated that HA was able to activate iDC, and that dendritic cells have improved capacity to present the mE7-mE6 antigen when said antigen is fused to HA.

TABLE 1
List of in vitro transcribed products (IVT mRNA)

| | Components | | | | |
|---|---|---|---|---|---|
| | HA | Linker | | Linker | |
| Construct name | subtype | 1 | E7 | 2 | E6 |
| mE7-mE6 | — | — | mE7 with | GS | mE6 |
| (SEQ ID NO: 7) | | | a signal | | |
| | | | peptide at | | |
| | | | N-terminus | | |
| tHA-mE7-mE6 (GS) | H1 | GS | mE7 | GS | mE6 |
| (SEQ ID NO: 8) | | | | | |
| tHA-mE7-mE6 (H4) | H1 | H4 | mE7 | H4 | mE6 |
| (SEQ ID NO: 9) | | | | | |
| tHA-mE7-mE6 | H1 | 2X(H4) | mE7 | 2X(H4) | mE6 |
| (2 × H4) | | | | | |
| (SEQ ID NO: 10) | | | | | |
| tHA-smnE7-smnE6 | H1 | GS | smnE7 | GS | smnE6 |
| (GS) | | | | | |
| (SEQ ID NO: 13) | | | | | |
| tHA-smnE7-smnE6 | H5 | GS | smnE7 | GS | smnE6 |
| (H5) | | | | | |
| (SEQ ID NO: 14) | | | | | |

### Example 4

Strong CD8⁺ T Cell-Specific Immune Response Induced by LNP-mRNA Expressing mE7-mE6 Fusion Protein

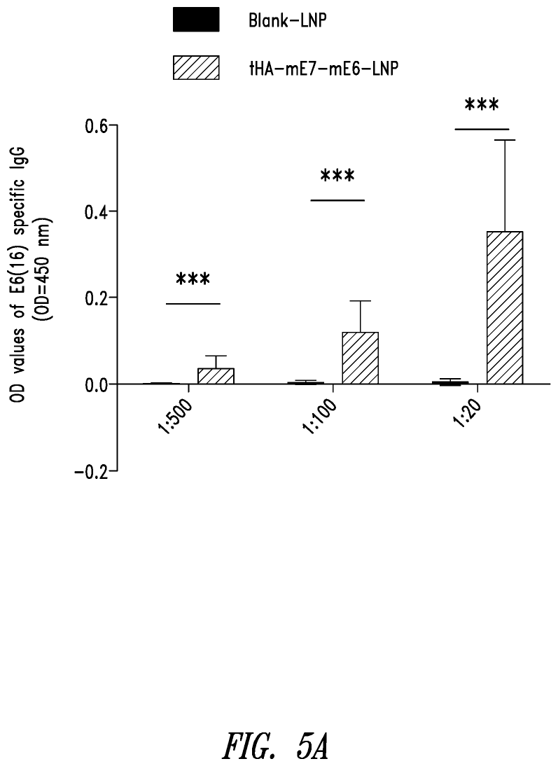

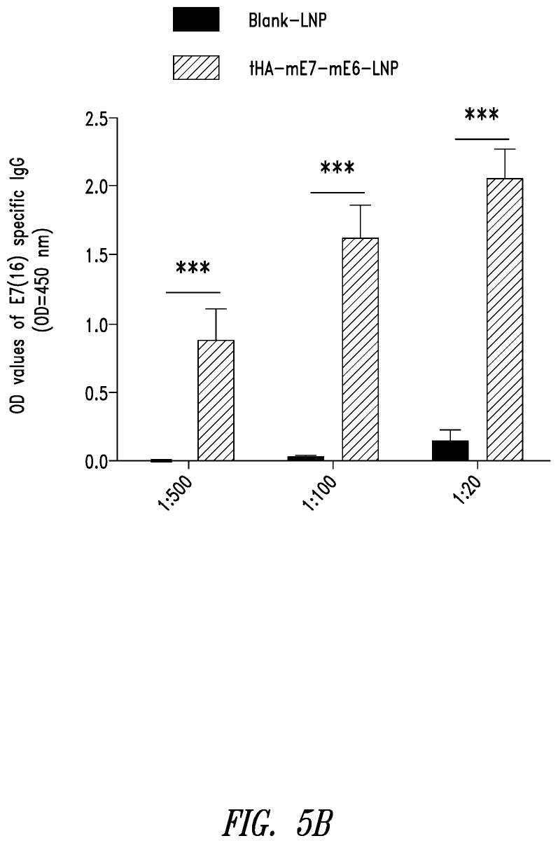

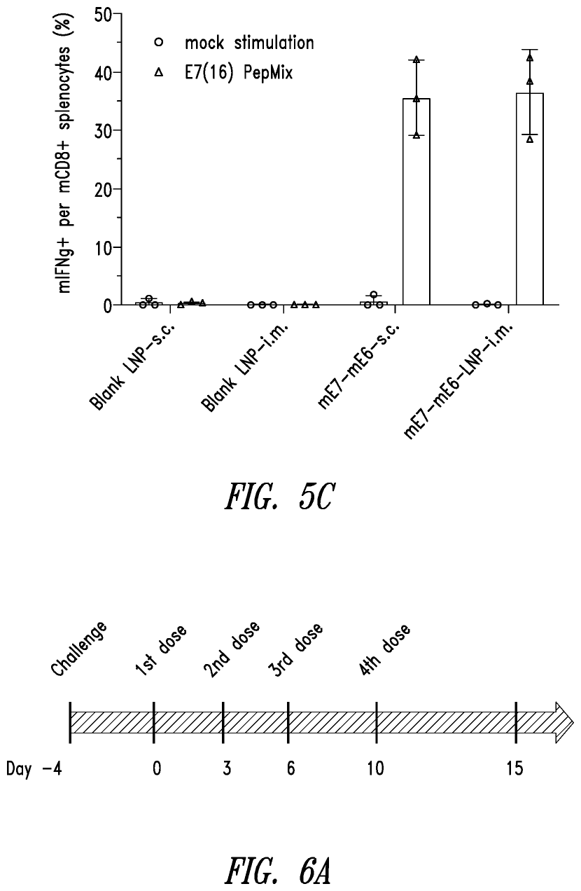

To test the HPV antigen-specific T cell immune response induced by an mRNA vaccine expressing the mE7-mE6 fusion protein, C57BL6 mice (6-8 weeks old) were vaccinated with 10⁹ pmol tHA-mE7-mE6-LNP (tHA-mE7-mE6 expressing mRNA encapsuled within lipid nanoparticles) three times with a 1-week interval between doses. Each dose contained 109 pmol mRNA in 50 μl PBS with sucrose. Mice were sacrificed one week after the final dose to harvest serum and spleen cells. Serum was used to detect total IgG against HPV antigen E6(16) and E7(16) using ELISA. The results indicated that tHA-mE7-mE6-LNP induced high levels of E6(16)-specific antibody and E7(16)-specific antibody in the serum, as shown in the graphs presented in FIGS. 5A and 5B, suggesting activation of humoral response. Spleen cells were harvested and co-cultured with a pool of E7(16) peptides (15 amino acids in length with 11 amino acid overlap). The percentage of mIFN-γ⁺ cells in the mCD8⁺ cell population was determined by flow cytometry. The results showed that tHA-mE7-mE6-LNP stimulated antigen-specific CD8⁺ T cell response, and that comparable T cell responses were induced through two routes of administration, subcutaneous (s.c) and intramuscular (i.m.), as shown in the graph presented in FIG. 5C.

### Example 5

mRNA-LNP with tHA-mE7-mE6 Induced Regression of E6-E7 Positive Tumor in a Syngeneic Mouse Tumor Model

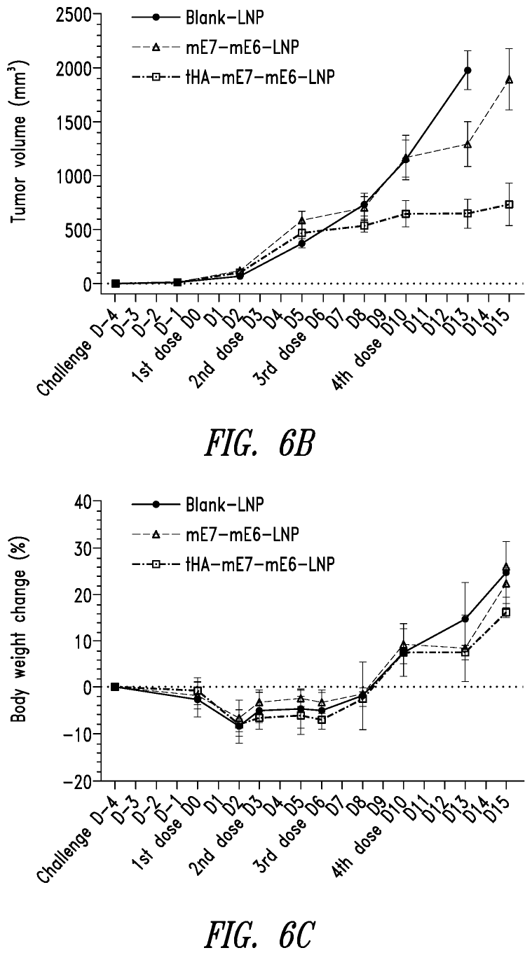

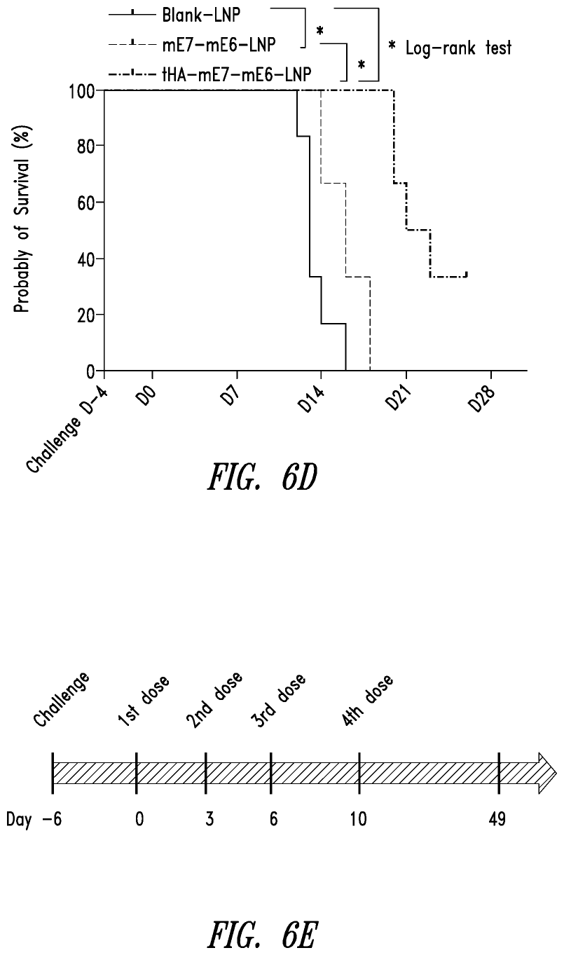

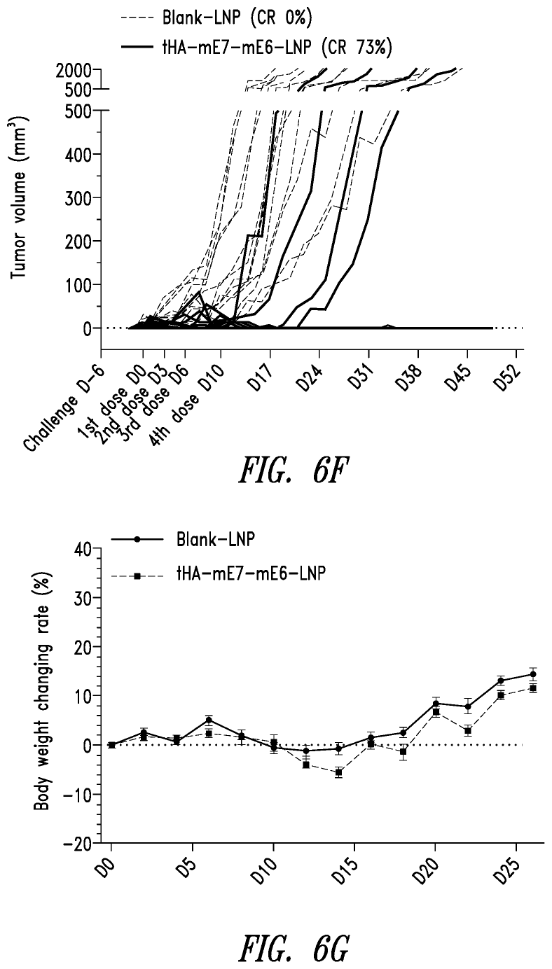

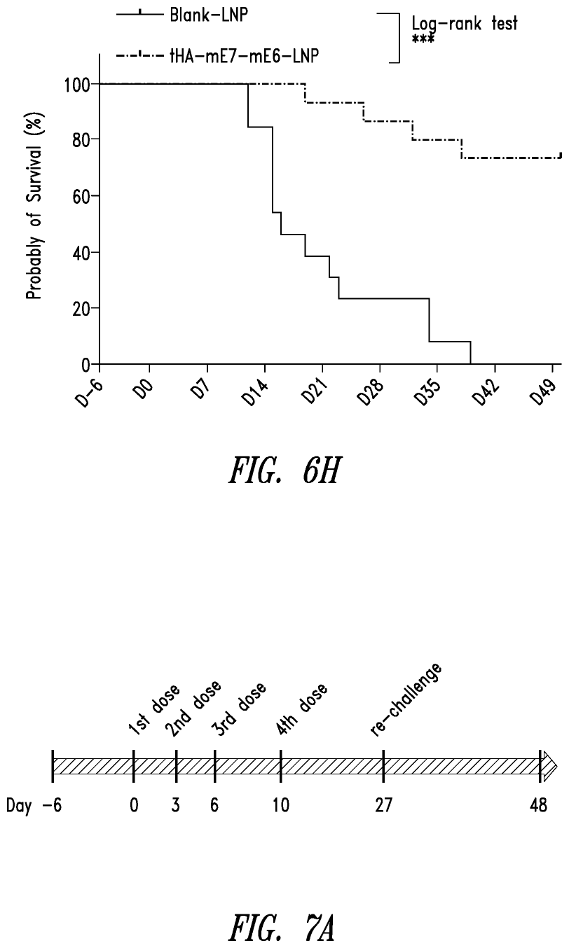

An E6- and E7-expressing cell line, TC1, was used to establish syngeneic subcutaneous tumor in mice. Mice challenged with high doses (5×10⁶ cells per dose) of TC1 were treated with indicated mRNA-LNP vaccines. The first dose was administered after all mice developed solid tumor bulges at the injection site, followed by 3 more doses, as indicated in FIG. 6A. The tumor growth was significantly suppressed in mE7-mE6- and tHA-mE7-mE6-LNP groups compared with the Blank-LNP, with a better therapeutic efficacy from tHA-mE7-mE6-LNP than mE7-mE6-LNP (see graph presented in FIG. 6B). After administrating the first two doses of therapy, mice in all groups showed slight drop in body weight, less than 10% (see graph presented in FIG. 6C), indicating that vaccine induced minor stress. Mice treated mE7-mE6-LNP had a longer survival time period than the control group, yet eventually all mice were sacrificed by day 18 for their tumor volumes reached ethical endpoint. In comparison, the tHA-mE7-mE6-LNP treated group had significantly longer lifespan and one out of six mice (17%) showed complete tumor regression after 4 doses of therapy (see graph presented in FIG. 6D).

Considering that it takes time for the mice to develop an anti-tumor immune response after receiving the mRNA-LNP therapy, the rapid progression of tumors that were inoculated with 5×10⁶ cells per dose limited the time available for the therapy to establish immunity. Thus, relatively slow-growing subcutaneous tumors were created by administrating 5×10⁴ cells per dose in mice to allow a longer therapeutic window. After all mice developed a solid tumor at injection site, four doses of mRNA-LNP were administrated with 2-3 days intervals, as indicated in FIG. 6E. Compared with Blank-LNP group, the tumor growth was significantly suppressed during and after the treatment of tHA-mE7-mE6-LNP. Eleven out of 15 mice (73%) achieved complete tumor regression post therapy (see graph presented in FIG. 6F). Minor body weight change was observed after vaccination (see graph presented in FIG. 6G). The survival rate of mice in tHA-mE7-mE6-LNP group was significantly higher than Blank-LNP group (see graph presented in FIG. 6H).

### Example 6

tHA-mE7-mE6-LNP Therapy Prevented Primary Formation and Recurrence of E6-E7 Positive Tumor

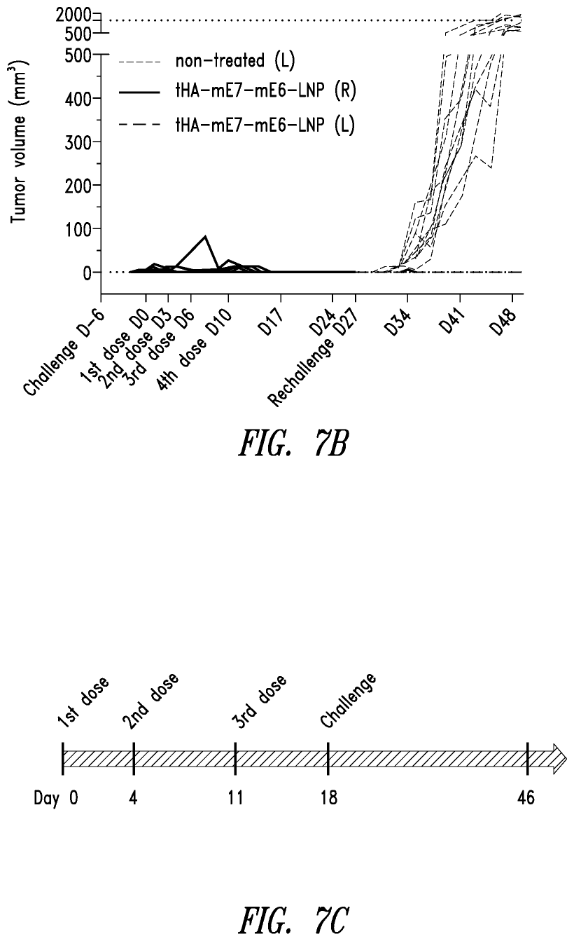

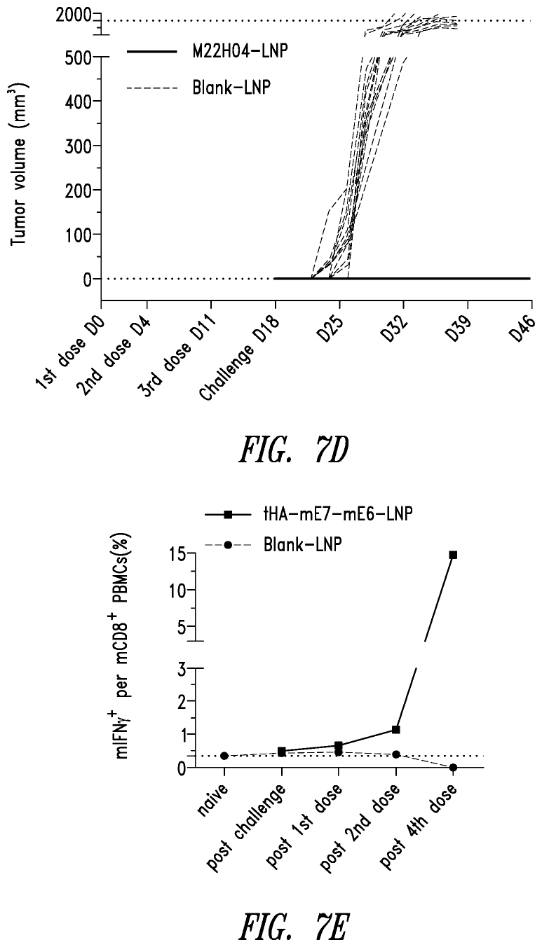

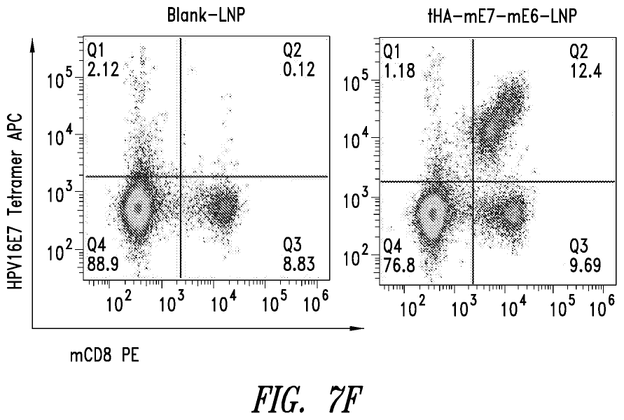

The mice recovered from TC1-cell challenge in FIGS. 6D-6H were subcutaneously re-challenged with TC1 cells (1×10⁵ cells per dose) at 17 days post the last dose of mRNA-LNP therapy, as indicated in FIG. 7A. None of the mice developed tumor on the re-challenge site (see graph presented in FIG. 7B), indicating that the therapy induced a lasting immune response to inhibit tumor recurrence. This protective immunity potentially included the immune response against neoantigens released during the TC1 tumor regression. To confirm tHA-mE7-mE6-LNP induced immunity was sufficient to prevent TC1 tumor formation, two groups of naïve mice were vaccinated with 3 doses of tHA-mE7-mE6-LNP or Blank-LNP, respectively, as indicated in FIG. 7C. None of the mice developed tumors after TC1 challenge (2×10⁵ cells per dose) in tHA-mE7-mE6-LNP treated group, confirming that the antigen-specific immunity raised by tHA-mE7-mE6-LNP was sufficient to prevent tumor formation (see graph presented in FIG. 7D).

To evaluate the antigen-specific CD8⁺ T cell response after each dose of tHA-mE7-mE6-LNP, peripheral blood mononuclear cells (“PBMCs”) were collected from tail-vein blood of mice and pooled in each group to test the activation level of CD8⁺ T cells post E7 PepMix stimulation. Blood from 6 mice were pooled at each time point for analysis. The percentage of mIFN-γ⁺ cells in mCD8⁺ cells were measured by flow cytometry at 6 hours post E7 PepMix stimulation. The results showed that E7-specific CD8⁺ T cell population was increased in the peripheral circulation after the 2nd dose of tHA-mE7-mE6-LNP and continued to increase with additional two doses of tHA-mE7-mE6-LNP (see graph presented in FIG. 7E). CD8⁺ T cells recognizing E7 epitope-loaded MHC-1-restricted tetramers were observed in the spleens of mice post the 4th dose of vaccine (see graph presented in FIG. 7F).

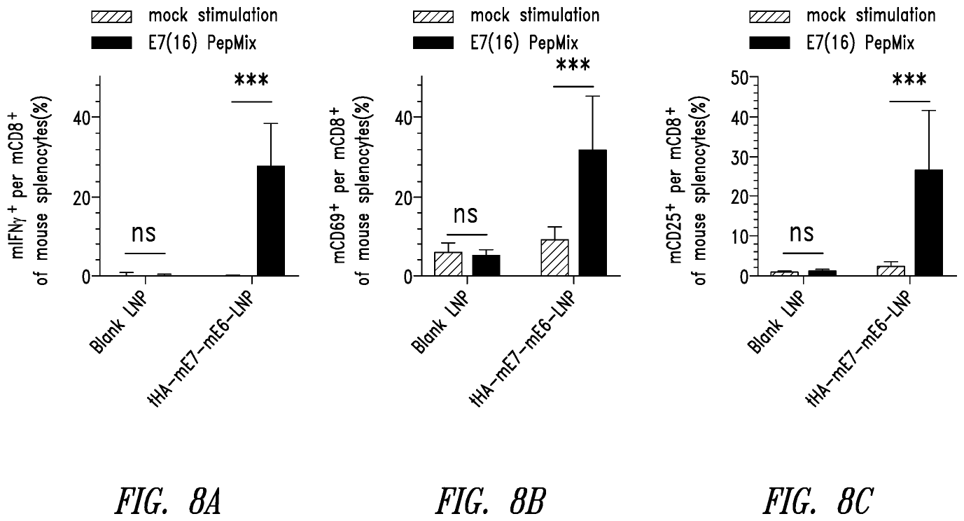

Spleens were collected at one week post the 4th dose of mRNA-LNP. In these mice, CD8⁺ T cells in spleen cell mixtures were activated by E7 PepMix and showed increased levels of mIFN-γ and mCD69 (see graphs presented in FIGS. 8A and 8B), indicating that E7-specific T cell responses were generated. The increased frequency of mCD25⁺ and mCD8⁺ T cells after stimulation suggests that these cells have a strong potential for proliferation (see graph presented in FIG. 8C). Several proteins associated with degranulation and killing capacity of CD8⁺ T cells, such as mCD63, mCD107a and mGranzyme B were found to be increased after E7 PepMix stimulation in tHA-mE7-mE6-LNP treated mice (see graphs presented in FIGS. 8D-8F).

### Example 7

tHA-mE7-mE6-LNP Therapy Enhanced Immune Cell Infiltration and Activation in Tumor

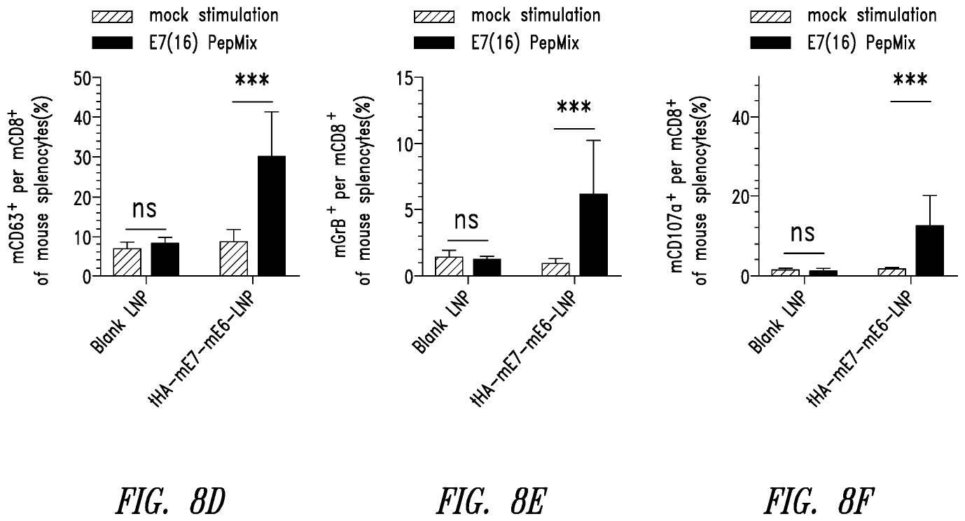

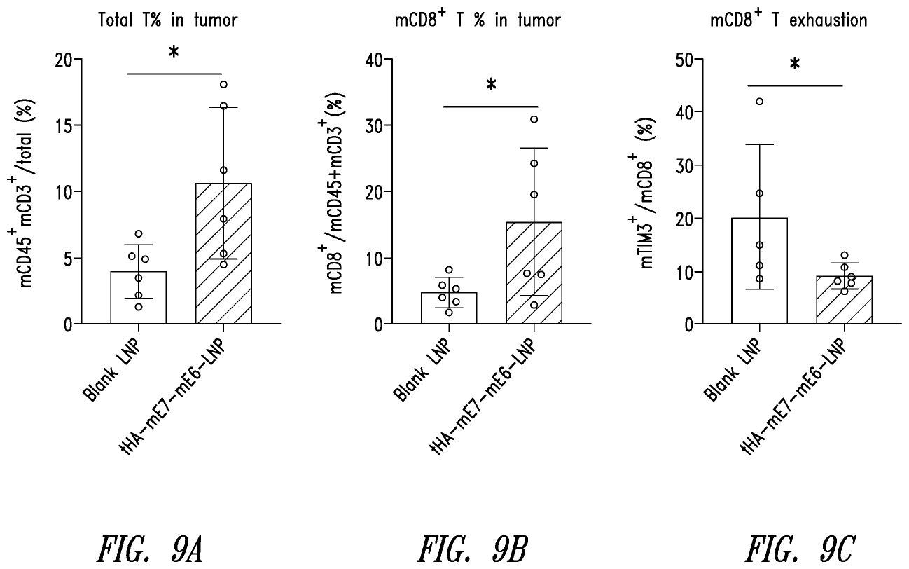

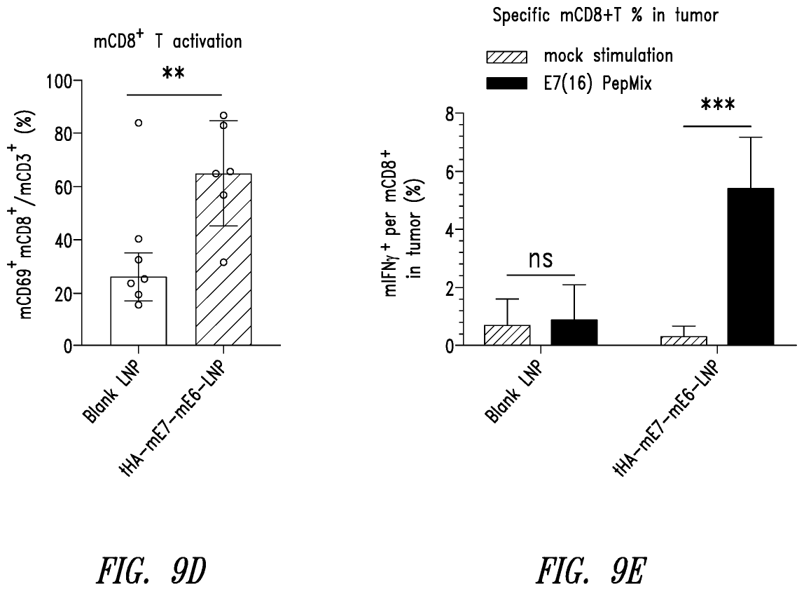

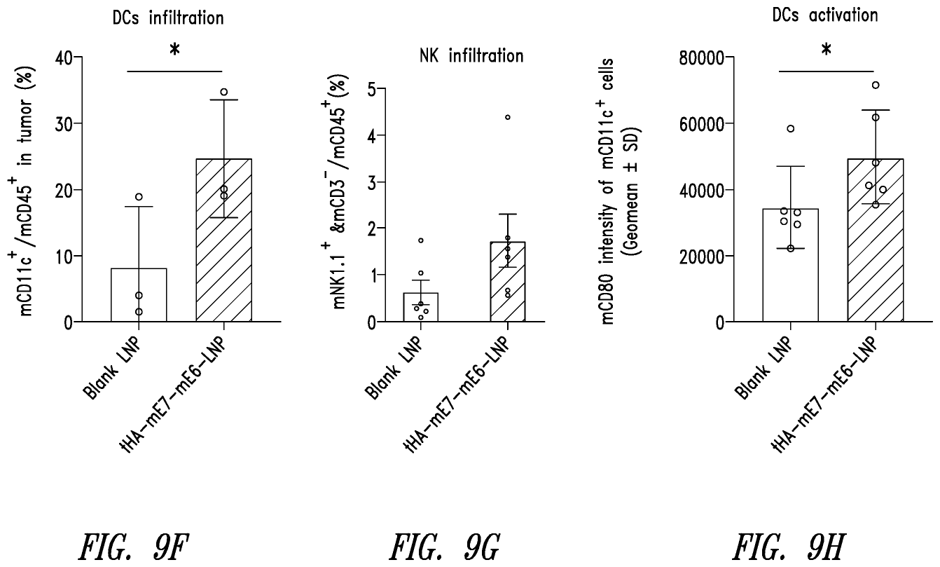

To evaluate the impact of intratumoral immunity of tHA-mE7-mE6-LNP therapy, the percentage of immune cell infiltrating into TC1 tumors were compared between Blank-LNP and tHA-mE7-mE6-LNP groups. After three doses of tHA-mE7-mE6-LNP therapy the percentage of mCD3⁺ and mCD45⁺ T cells in tumor were increased by three folds, comparing with the Blank-LNP group (see graph presented in FIG. 9A). The percentage total mCD8⁺ T cells as well as E7 (PepMix)-specific mCD8⁺ T cells were increased in the tumor after tHA-mE7-mE6-LNP therapy (see graphs presented in FIGS. 9B and 9E). Since T cell exhaustion in tumor micro-environment plays one of the major roles in the failure of anti-cancer immunity, the exhaustion level of mCD8⁺ T cells were assessed by measuring mTIM3⁺ cells. The results showed that after tHA-mE7-mE6-LNP therapy, the population of mTIM3⁺ and mCD8⁺ T cells remained at a lower level than in the Blank-LNP treated tumors (see graph presented in FIG. 9C). The activation level of mCD8⁺ T cells were assessed by measuring mCD69⁺ and mCD8⁺ cells in mCD3⁺ T cell population showing that the percentage significantly increased aftert HA-mE7-mE6-LNP therapy (see graph presented in FIG. 9D). In addition, mCD45⁺ and mCD11c⁺ cells (FIG. 9F) and mCD45⁺, mCD3- and mNK1.1⁺ cells (FIG. 9H) were increased in the tumor micro-environment post tHA-mE7-mE6-LNP therapy, suggesting DCs and NK cells infiltrating into the tumor was enhanced. The increased mCD80 intensity on the surface of tumor infiltrated mCD11c⁺ cells in tHA-mE7-mE6-LNP group indicated that the therapy enhanced activation level of DCs (see graph presented in FIG. 9G).

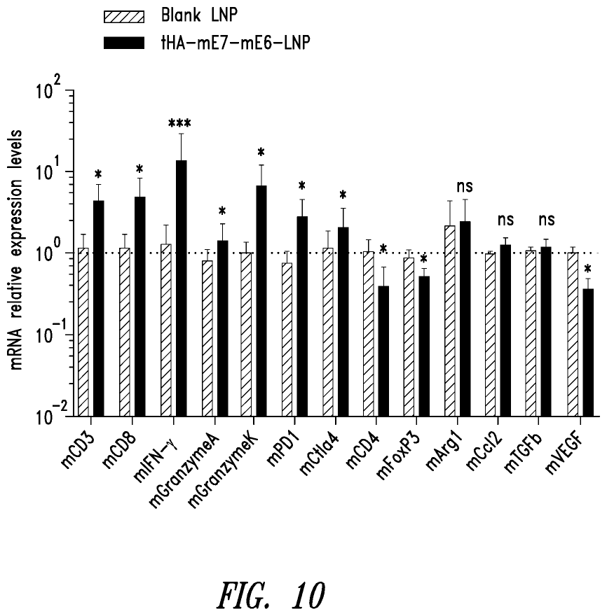

The expression of immune genes within the tumor were monitored with RT-qPCR using total RNA extracted from tumor tissue. The results in FIG. 10 showed increased expression of mCD3, mCD8, mIFN-γ, mGranzymes, mPD1 and mCTLA4 genes and supported that tHA-mE7-mE6-LNP vaccine increased CD8⁺ T cell immune response. The regulatory T (T reg)-related genes, mCD4 and mFoxP3, were reduced post vaccination. No significant changes were found in mArg1, an immunosuppressive mediator produced by myeloid-derived suppressor cells (MDSCs), mCCL2, a gene important for MDSC recruiting, and in mTGF3, a gene mostly played a suppressive role in anti-cancer immunity. The reduction of mVEGF transcription possibly was a result of tumor regression induced by tHA-mE7-mE6-LNP.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An immunogenic protein, comprising an amino acid sequence derived from one or more proteins which are responsible for cell proliferation, and wherein said one or more proteins are derived from an HPV strain associated with causing cancer.

2. The protein of claim 1, wherein said HPV strain associated with causing cancer is selected from the group consisting of HPV strain 16, 18, 31, 33, 35, 39, 45, 51, 52, 56, 58, 59, 66 or 68.

3. The protein of claim 1 or 2, wherein said protein is mutated such that it does not cause cell proliferation.

4. The protein according to claim 3 wherein said mutation is one or more amino acid changes selected from the group consisting of substitutions, insertions and deletions.

5. The protein according to claim 3 wherein said mutation eliminates or diminishes interactions with p53/pRB.

6. The protein according to claim 3 wherein said mutation reduces or prevents nuclear localization.

7. The protein according to claim 3 wherein said mutation does not substantively decrease the immune response as compared to unmutated E6 and E7.

8. The protein according to claim 3 wherein said mutated protein is E6 or E7.

9. An immunogenic fusion protein, comprising a protein according to any one of claims 1 to 8, and a cell-targeting moiety.

10. The immunogenic fusion protein according to claim 9, wherein said cell targeting moiety is all or a portion of an HA protein.

11. The immunogenic fusion protein according to claim 9 wherein said cell-targeting moiety is selected from the group consisting of Ebola virus glycoprotein, envelope glycoprotein gp120 of HIV, Marburg virus glycoprotein, E2 glycoprotein from hepatitis C virus, and the spike protein of COVID19.

12. An immunogenic fusion protein comprising:
a first amino acid sequence having at least 80% sequence identity with SEQ ID NO:3 (mE6(16)), and
a second amino acid sequence having at least 80% sequence identity with SEQ ID NO:4 (mE7(16)).

13. An immunogenic fusion protein comprising:
a first amino acid sequence having at least 80% sequence identity with SEQ ID NO:5 (smnE6(16)), and
a second amino acid sequence having at least 80% sequence identity with SEQ ID NO:6 (smnE7(16)).

14. An immunogenic fusion protein, comprising a sequence according to SEQ ID. NO. 7.

15. An immunogenic fusion protein, comprising a sequence according to SEQ ID. NO. 8.

16. An immunogenic fusion protein, comprising a sequence according to SEQ ID. NO. 9.

17. An immunogenic fusion protein, comprising a sequence according to SEQ ID. NO. 10.

18. An immunogenic fusion protein, comprising a sequence according to SEQ ID. NO. 11.

19. An immunogenic fusion protein, comprising a sequence according to SEQ ID. NO. 12.

20. An immunogenic fusion protein, comprising a sequence according to SEQ ID. NO. 13.

21. An immunogenic fusion protein, comprising a sequence according to SEQ ID. NO. 14.

22. An isolated nucleic acid sequence which encodes an immunogenic protein or immunogenic fusion protein according to any one of claims 1 to 21.

23. The isolated nucleic acid sequence according to claim 22 wherein said nucleic acid sequence is an mRNA sequence.

24. An expression cassette which directs the expression of an immunogenic protein or immunogenic fusion protein according to any one of claims 1 to 21.

25. A composition comprising an mRNA sequence encoding an immunogenic protein or immunogenic fusion protein according to any of claims 1 to 21.

26. The composition according to claim 25, wherein said composition is contained within a nanoparticle or liposome.

27. A pharmaceutical composition, comprising an immunogenic protein or immunogenic fusion protein according to any one of claims 1 to 21.

28. A pharmaceutical composition comprising an isolated nucleic acid sequence according to claim 22 or 23.

29. A pharmaceutical composition comprising an expression cassette according to claim 24.

30. A pharmaceutical composition comprising an mRNA sequence according to claim 25 or claim 26.

31. A vaccine which induces an immune response in a subject, comprising a pharmaceutically acceptable vehicle and a pharmaceutically effective amount of an immunogenic protein or immunogenic fusion protein according to any one of claims 1 to 21.

32. A vaccine which induces an immune response in a subject, comprising a pharmaceutical composition according to claim 30.

33. A method of treating an HPV-related disease in a subject in need thereof, comprising administering a therapeutically effective amount of a vaccine according to claim 31 or 32, to a subject.

34. The method according to claim 33, wherein the HPV-related disease is cancer.

35. The method according to claim 34, wherein the HPV-related disease is HPV-positive cancer.

36. The method according to claim 35, wherein the cancer comprises adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor.

37. The method according to claim 33, wherein the HPV-related disease is a precancerous condition, or, recurrent respiratory papillomatosis (RRP).

38. The method according to claim 37, wherein the precancerous condition comprises cervical dysplasia or squamous intraepithelial lesion, prostatic intraepithelial neoplasia, superficial bladder cancer, precancerous lesions of the breast, precancerous lesions of the lung, actinic keratosis, Barrett's esophagus, precancerous melanoma moles, precancerous conditions of the uterus/vulva, precancerous conditions of the ovary, atrophic gastritis, precancerous conditions of the oral cavity, general dysplastic conditions, squamous metaplasia, intraepithelial neoplasia, precancerous conditions in the head or neck, familial adenomatous polyposis (Gardner's syndrome), sporadic adenomatous polyposis, hereditary non-polyposis colon cancer (Lynch syndrome), and Crohn's disease.

39. A method of treating HPV in a subject in need thereof, comprising administering a therapeutically effective amount of a vaccine according to claim 31 or 32, to a subject.

40. The method according to any one of claims 33 to 39, wherein the vaccine is administered to the subject intratumorally, subcutaneously, intramuscularly, intradermally or intravenously.


SEQUENCE LISTING (14 sequences)

SEQ ID NO: 1 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 158 aa, Human papillomavirus type 16
MHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 4 - protein, 98 aa, Human papillomavirus type 16
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 5 - protein, 158 aa, Human papillomavirus type 16
MHQKRTAMFQDPQEGPGELPQLCTELQTTIHDIILECVYCKQQLLRREVYDFAFRDGCIVYRDGNPYAVCDECLEFYSEISEYRHYCYSLYGTTLEQQYNKPLCDLLIRCINRQKPLCPEEEQNDLDNEQRFHNIRGRWTGRCMSCCRSSRTRRETQL

SEQ ID NO: 6 - protein, 98 aa, Human papillomavirus type 16
MHGDTPTLHEYMLDLQPETTDLYGYGQLNDSSEEEDEIDGPAGQAEPDRAHYNIVTFCCKCDSTARACAQSTHVDIRTLEDLLMGTLGIVCPICSQKP

SEQ ID NO: 7 - protein, 280 aa, Human papillomavirus type 16
MKANLLVLLCALAAADAHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGSGGGGGMHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 8 - protein, 801 aa, Human papillomavirus type 16
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQGGSGGGGGMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGSGGGGGMHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 9 - protein, 837 aa, Human papillomavirus type 16
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQLEAEAAAKEAAAKEAAAKEAAAKALEMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPLEAEAAAKEAAAKEAAAKEAAAKALEMHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 10 - protein, 889 aa, Human papillomavirus type 16
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQLEAEAAAKEAAAKEAAAKEAAAKALELEAEAAAKEAAAKEAAAKEAAAKALEMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPLEAEAAAKEAAAKEAAAKEAAAKALELEAEAAAKEAAAKEAAAKEAAAKALEMHQKRTAMFQDPQERPRKLPQLCTELQTTIHDIILECVYCKQQLLRREVYDFAFRDGCIVYRDGNPYAVCDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 11 - protein, 801 aa, Human papillomavirus type 16
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQGGSGGGGGMHGDTPTLHEYMLDLQPETTDLYGYGQLNDSSEEEDEIDGPAGQAEPDRAHYNIVTFCCKCDSTARACAQSTHVDIRTLEDLLMGTLGIVCPICSQKPGGSGGGGGMHQKRTAMFQDPQEGPGELPQLCTELQTTIHDIILECVYCKQQLLRREVYDFAFRDGCIVYRDGNPYAVCDECLEFYSEISEYRHYCYSLYGTTLEQQYNKPLCDLLIRCINRQKPLCPEEEQNDLDNEQRFHNIRGRWTGRCMSCCRSSRTRRETQL

SEQ ID NO: 12 - protein, 803 aa, Human papillomavirus type 16
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQGGSGGGGGMHGDTPTLHEYMLDLQPETTDLYGYGQLNDSSEEEDEIDGPAGQAEPDRAHYNIVTFCCKCDSTARACAQSTHVDIRTLEDLLMGTLGIVCPICSQKPGGSGGGGGMHQKRTAMFQDPQEGPGELPQLCTELQTTIHDIILECVYCKQQLLRREVYDFAFRDGCIVYRDGNPYAVCDECLEFYSEISEYRHYCYSLYGTTLEQQYNKPLCDLLIRCINRQKPLCPEEEQNDLDNEQRFHNIRGRWTGRCMSCCRSSRTRRETQL

SEQ ID NO: 13 - protein, 801 aa, Homo sapiens; identical to SEQ ID NO: 11

SEQ ID NO: 14 - protein, 803 aa, Homo sapiens; identical to SEQ ID NO: 12